(19) United States
(12) Patent Application Publication
(10) Pub. No.: US 2020/0098482 A1
(43) Pub. Date: Mar. 26, 2020

(54) INTERACTIVE DEVICE FOR DISPLAYING PERSONALIZED CONTENT FOR AIDING IN THE CARE OF ASSISTED LIVING

(71) Applicants: Michael Robert Groves, Elkridge, MD (US); Stuart Lowry, Elkridge, MD (US)

(72) Inventors: Michael Robert Groves, Elkridge, MD (US); Stuart Lowry, Elkridge, MD (US)

(21) Appl. No.: 16/577,775

(22) Filed: Sep. 20, 2019

Related U.S. Application Data
(60) Provisional application No. 62/735,921, filed on Sep. 25, 2018.

(51) Int. Cl. G16H 80/00; G06F 3/02; G06F 3/147; H01H 13/14; H01H 13/02

(52) U.S. Cl. CPC G16H 80/00; G06F 3/02; H01H 13/023; H01H 13/14; G06F 3/147

(57) ABSTRACT

A wall-mountable interactive device for displaying messages with photos and personalized information is provided. The large size of the display of the interactive device as well as the prominently presented colored buttons facilitates in providing living assistance to individuals in need of assisted living yet live independently. Each colored button provides straight forward functionality for the individual to communicate with a remote caregiver by way of wireless local area networking.

### CROSS-REFERENCE TO RELATED APPLICATION

This application claims the benefit of priority of U.S. provisional application No. 62/735,921, filed 25 Sep. 2018, the contents of which are herein incorporated by reference.

### BACKGROUND OF THE INVENTION

The present invention relates to electronic interactive devices and, more particularly, a wall-mountable interactive device employing wireless local area networking for displaying messages with photos and personalized information, and the method aiding in the caregiving of individuals in need of assisted living.

Lack of communication between a caregiver and an individual in need of assisted living, when that individual is living independently, can be very challenging and risks severe and possibly dangerous consequences. Though for those you cannot afford round-the-clock assisted living, such situations are not uncommon. Electronic user interfaces are helpful in solving the challenges, but current touchscreen-based devices are small, hard to read and complicated to use. Moreover, non-technical users or older adults can easily get confused with swipe and touch functionality.

As can be seen, there is a need for a wall-mountable interactive device employing wireless local area networking for displaying messages with photos, personalized information, and real-time messaging/reminders through a large non-touch screen, with easy to read text, lighted buttons, and a simple push button interface and functionality. Thereby, enabling a method for aiding in the caregiving of individuals in need of assisted living, though the individuals are living alone.

### SUMMARY OF THE INVENTION

In one aspect of the present invention, a method of aiding in the caregiving for an individual in need of assisted living, includes the following: providing an interactive device for displaying content; the interactive device having a display surface of at least two square feet in area; a plurality of indicia buttons disposed adjacent the display surface, wherein each indicia button enables a different communicative functionality to communicate an individual in need of assisted living and a remote caregiver; and mounting the interactive device along a wall that is visible for the individual, wherein the plurality of indicia buttons include a first-indicium button, a second-indicium button and a third-indicium button, wherein deploying the third-indicium button displays one or more archived messages on the display surface, wherein deploying the second-indicium button displays one or more archived images on the display surface, and wherein deploying the third-indicium button alerts the remote caregiver, wherein deploying the first-indicium and second-indicium buttons simultaneously enables a setup mode. The remote caregiver can program the interactive device to switch from a wake mode to a sleep mode after a predetermined time without deployment of any of the plurality of indicia buttons; and providing a motion sensor operatively associated with the interactive device, wherein motion sensed by the motion sensor switches the interactive device to the wake mode from the sleep mode, wherein each archived message or image is displayed on a selective order, wherein the selective order is sequential in time.

These and other features, aspects and advantages of the present invention will become better understood with reference to the following drawings, description and claims.

### BRIEF DESCRIPTION OF THE DRAWINGS

FIG. 1 is a perspective view of an exemplary embodiment of the present invention, demonstrating illumination of a plurality of colored light-emitting elements;

FIG. 2 is a front view of an exemplary embodiment of the present invention, demonstrating illumination of the plurality of colored light-emitting elements;

FIG. 3 is an assembled view of an exemplary embodiment of internal components of the present invention;

FIG. 4 is a flowchart of an exemplary embodiment of the construction of the present invention; and

FIG. 5 is a flowchart of an exemplary embodiment of the functions of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a wall-mountable interactive device for displaying messages with photos and personalized information. The large size of the display of the interactive device as well as the prominently presented colored buttons facilitates in providing living assistance to individuals in need of assisted living yet live independently. Each colored button provides straight forward functionality for the individual to communicate with a remote caregiver by way of wireless local area networking.

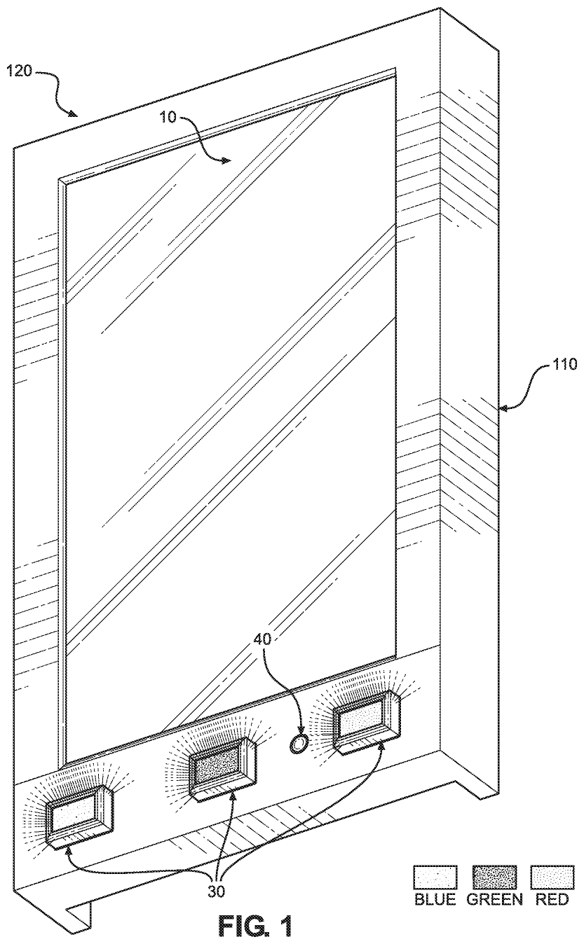

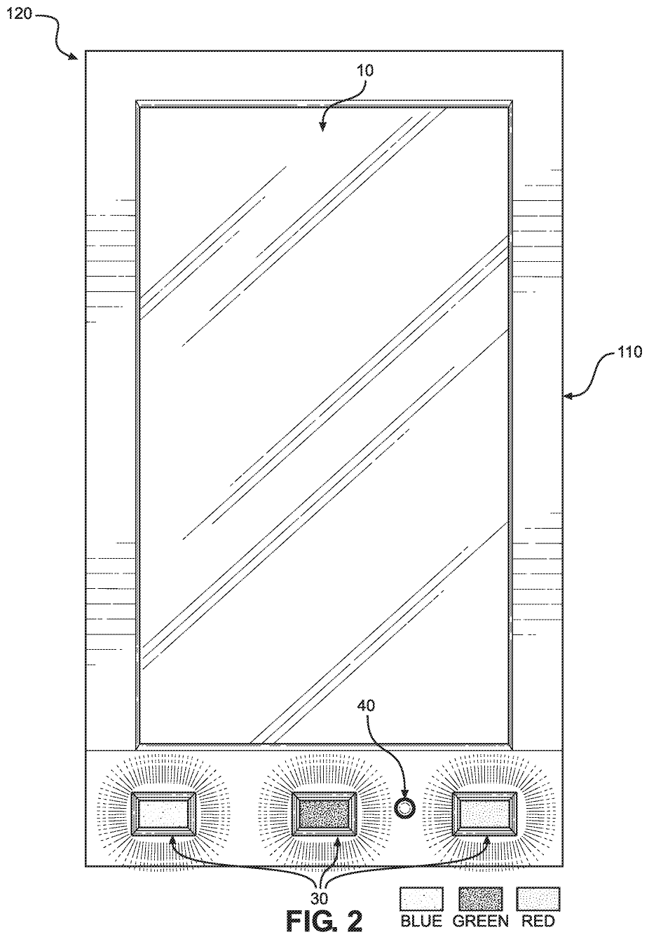

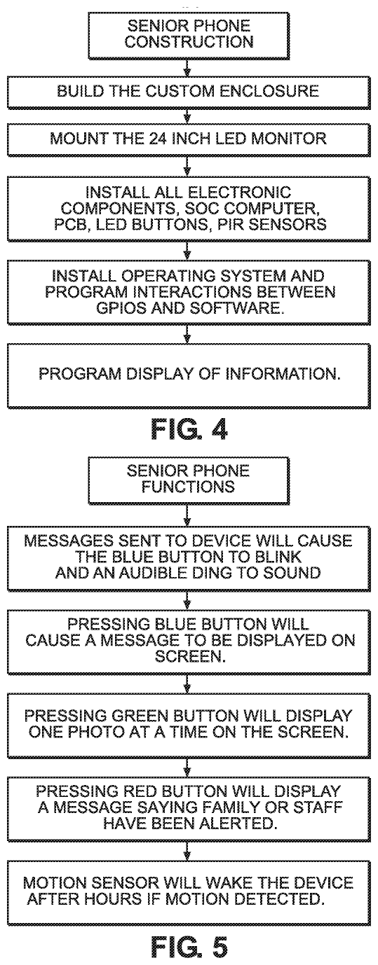

Referring to FIGS. 1 through 5, the present invention may include at least one computer with a user interface/display 10. The computer 20 may include at least one processing unit operatively coupled to a form of memory including, but not limited to, a server, microprocessor, a desktop, laptop, and smart device, such as, a tablet and smart phone. The computer 20 includes a program product including a machine-readable program code for causing, when executed, the computer 20 to perform steps. The program product may include software which may either be loaded onto the computer or accessed by the computer 20. The loaded software may include an application on a smart device. The software may be accessed by the computer 20 using a web browser. The computer 20 may access the software via the web browser using the internet, extranet, intranet, host server, internet cloud and the like.

The wall-mountable interactive display device 120 may embody the computer 20 including a custom fabricated PCB 50 coupled to the large LCD display/interface 10 to represent content. In certain embodiments, this may include an LCD display 10 operatively coupled (e.g., via a HDMI cable 80) to an arm-based SOC computer 20 with wireless local area networking capabilities (e.g., Wi-Fi™) and HDMI output, wherein the arm-based SOC computer 20 provides HDMI input and built in speakers. A power supply 70, for instance 110v and 5v outputs, may be included for powering the large LCD display 10 and the arm-based SOC computer 20.

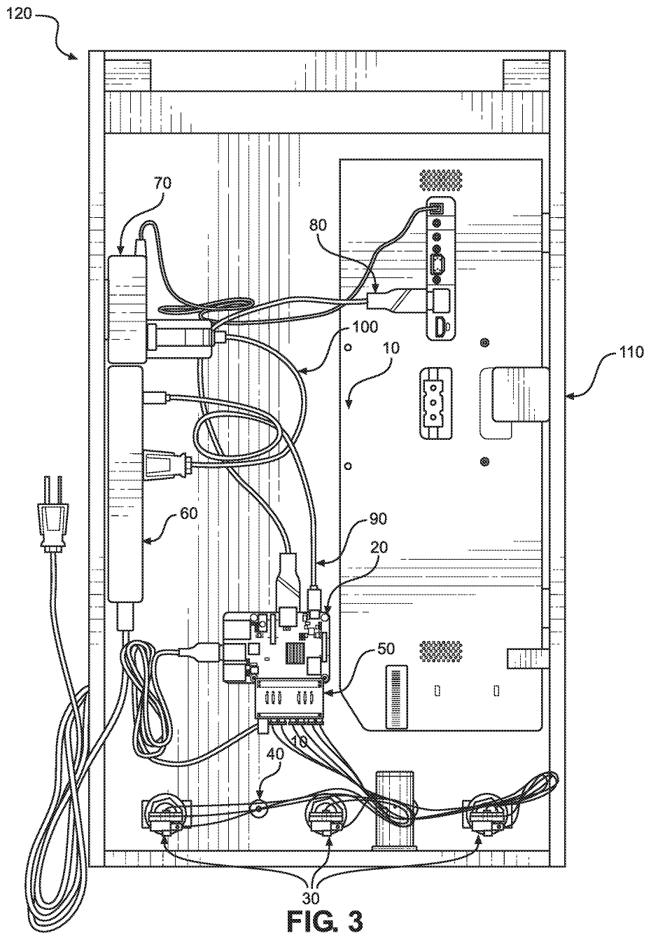

The power supply 70 may be coupled to a power strip 60 with surge protectors by way of a power cable 100. The computer 20 may be housed in a custom fabricated enclosure 110 to physically secure the monitor 10 and components, exemplarily illustrated in FIG. 3. The enclosure 110 may provide a plurality of external LED lighted buttons 30, a PIR motion sensor 40, and a megapixel camera (for setup, for instance).

In certain embodiments, the custom fabricated PCB 50 may operatively couple the plurality of external LED lighted buttons 30 and the PIR motion sensor 40 to the arm-based SOC computer 20 for providing input and output. The megapixel camera may connect via a USB cable 90 to the arm-based SOC computer 20 for providing video input.

The wall-mountable interactive display device 120, colloquially known as the “senior phone”, is programmable to connect to Internet-based, systemic servers for pulling any updated information to be displayed on the user-interface/monitor display 10. Any new messages will be downloaded to the wall-mountable interactive display device 120, enabling visual and audible notifications (via the lighted buttons 30 and/or audible output by way of speakers or other audible output devices).

Pressing one or more of the plurality of LED buttons 30 may result in predetermined functionality. Pressing the blue button, for instance, may display a message. Pressing the green button may display photos/images one at a time. Pressing the red button may cause an alert message to be sent to caregivers. All button presses and motion detected is logged and uploaded to the systemic servers, and possibly governed by the following set of rules:

If the blue button is pressed, any new or archived messages will be displayed in a predetermined order. The predetermined order may be sequentially in time, by a selected priority, or the like. It has been found by the inventor that archiving messages, as opposed to continuously or real-time communication of messages increases the likelihood of an average individual in need of assisted living will both receive and retain the message.

If the green button is pressed, any images/photos may be displayed in a predetermined order.

If the red button is pressed, a notification message may be generated to remote caregiver(s).

If motion is detected via the PIR motion sensor 40, activity will be logged and night-mode will be turned off for a determined amount of time.

If both blue and red buttons are pressed together, the setup function will execute.

If a message was received, the blue light will blink and an audible notification may be played.

A method of manufacturing the present invention may include the following. A manufacturer could build a custom enclosure 110, then install and connect all the components in the enclosure 110. Then the manufacturer or distributor could download and install the operating system, e.g., Linux Operating System, and install the systemic custom code and execute it. The arm-based SOC computer 20 may be required to access content from the systemic Internet-based servers and to represent that content to the monitor/screen 10. The camera and motion sensor 40 are not required for daily operations. The LED buttons 30 are required to allow user interaction with the interactive display device 120.

A method of using the present invention may include the following. The interactive display device 120 disclosed above may be provided. A caregiver would install/setup the interactive display device 120 in the user's home, in a central location easily accessible to user, for instance mounted on a wall easily viewable by the individual in need of assisted living. The caregiver will then be able to send messages, update user's content and set reminders. User will be able to see the content, calendar and messages, and use the simple button interface to interact with the “senior phone”.

Additionally, the present invention could be used in an office environment to present current information and/or notices to staff/customers.

The computer-based data processing system and method described above is for purposes of example only, and may be implemented in any type of computer system or programming or processing environment, or in a computer program, alone or in conjunction with hardware. The present invention may also be implemented in software stored on a computer-readable medium and executed as a computer program on a general purpose or special purpose computer. For clarity, only those aspects of the system germane to the invention are described, and product details well known in the art are omitted. For the same reason, the computer hardware is not described in further detail. It should thus be understood that the invention is not limited to any specific computer language, program, or computer. It is further contemplated that the present invention may be run on a stand-alone computer system, or may be run from a server computer system that can be accessed by a plurality of client computer systems interconnected over an intranet network, or that is accessible to clients over the Internet. In addition, many embodiments of the present invention have application to a wide range of industries. To the extent the present application discloses a system, the method implemented by that system, as well as software stored on a computer-readable medium and executed as a computer program to perform the method on a general purpose or special purpose computer, are within the scope of the present invention. Further, to the extent the present application discloses a method, a system of apparatuses configured to implement the method are within the scope of the present invention.

It should be understood, of course, that the foregoing relates to exemplary embodiments of the invention and that modifications may be made without departing from the spirit and scope of the present invention.

What is claimed is:

1. A method of aiding in the caregiving for an individual in need of assisted living, comprising:
providing an interactive device for displaying content;
the interactive device having a display surface of at least two square feet in area;
a plurality of indicia buttons disposed adjacent the display surface, wherein each indicia button enables a different communicative functionality to communicate an individual in need of assisted living and a remote caregiver; and
mounting the interactive device along a wall that is visible for the individual.

2. The method of claim 1, the plurality of indicia buttons include a first-indicium button, a second-indicium button and a third-indicium button, wherein deploying the third-indicium button displays one or more archived messages on the display surface, wherein deploying the second-indicium button displays one or more archived images on the display surface, and wherein deploying the third-indicium button alerts the remote caregiver.

3. The method of claim 1, wherein deploying the first-indicium and second-indicium buttons simultaneously enables a setup mode.

4. The method of claim 2, further comprising programming the interactive device to switch from a wake mode to a sleep mode after a predetermined time without deployment of any of the plurality of indicia buttons; and
providing a motion sensor operatively associated with the interactive device, wherein motion sensed by the motion sensor switches the interactive device to the wake mode from the sleep mode.

5. The method of claim 2, wherein each archived message or image is displayed on a selective order, wherein the selective order is sequential in time.
